(19) United States
(12) Patent Application Publication
(10) Pub. No.: US 2026/0227371 A1
(43) Pub. Date: Aug. 6, 2026

(54) METHOD FOR PROVIDING FLAVOR EVALUATION, INFORMATION PROCESSING APPARATUS, FLAVOR INFORMATION PROVIDING SYSTEM, AND NON-TRANSITORY COMPUTER READABLE MEDIUM

(71) Applicant: SHIMADZU CORPORATION, Kyoto-shi, Kyoto (JP)

(72) Inventor: Keiko MATSUMOTO, Kyoto-shi, Kyoto (JP)

(73) Assignee: SHIMADZU CORPORATION, Kyoto-shi, Kyoto (JP)

(21) Appl. No.: 19/150,507

(22) PCT Filed: Jan. 23, 2024
(86) PCT No.: PCT/JP2024/001818

§ 371 Date: Jul. 23, 2025

(30) Foreign Application Priority Data
Jan. 26, 2023 (JP) 2023-010247

(51) Int. Cl. G01N 30/86; G01N 33/02

(52) U.S. Cl. CPC G01N 30/8675; G01N 33/02

(57) ABSTRACT

The present disclosure provides a method for providing an evaluation of a flavor of two or more food products.

### TECHNICAL FIELD

The present disclosure relates to providing an evaluation of the flavor of food products.

### BACKGROUND ART

Conventionally, various proposals have been made regarding the evaluation of the flavor of food products. In the field of food product manufacturing and development, specific evaluators assess the taste of prototypes, and Non-Patent Literature 1 discloses a method for training such evaluators (panels).

### PRIOR ART DOCUMENTS

### Non-Patent Literature

[Non-Patent Literature 1] Hideko Furukawa, “Panel Training and Product Development,” Journal of the Brewing Society of Japan, Japan, Brewing Society of Japan, Jun. 15, 1983, Vol. 78, No. 6, pp. 419-422.

### SUMMARY OF THE INVENTION

### Problem to be Solved by the Invention

With respect to the evaluations described above, there has been a demand for a technology to compare evaluations of a plurality of food products when such evaluations are made.

The present disclosure has been conceived in view of such circumstances, and an object thereof is to provide a technology for easily performing a comparative evaluation of the flavor of a plurality of food products.

### Means for Solving the Problem

A method according to one aspect of the present disclosure is a method for providing flavor information regarding two or more food products, the method comprising: a step of acquiring an analysis result of components of each of the two or more food products; a step of identifying a substance that affects a difference in flavor among the two or more food products, using a result of a test that uses the analysis result of the components of each of the two or more food products; and a step of outputting flavor information regarding the two or more food products, using the analysis result of the identified substance among the analysis results of the components of each of the two or more food products.

An information processing apparatus according to another aspect of the present disclosure comprises: one or more processors; and a storage device storing a program that, when executed by the one or more processors, causes the one or more processors to perform the method described above.

A flavor information providing system according to still another aspect of the present disclosure comprises: the information processing apparatus described above; and an analysis apparatus that outputs an analysis result of a food product to the information processing apparatus described above.

A program according to still another aspect of the present disclosure, when executed by one or more processors, causes the one or more processors to perform the method described above.

### Effects of the Invention

According to one aspect of the present disclosure, it becomes easy to perform a comparative evaluation of the flavor of a plurality of food products.

### BRIEF DESCRIPTION OF THE DRAWINGS

FIG. 1 is a diagram showing a configuration of a flavor information providing system 1.

FIG. 2 is a diagram showing an example of flavor information output by an information processing apparatus 100.

FIG. 3 is a diagram showing an example of flavor information output by the information processing apparatus 100.

FIG. 4 is a diagram showing an example of flavor information output by the information processing apparatus 100.

FIG. 5 is a diagram showing an example of flavor information output by the information processing apparatus 100.

FIG. 6 is a diagram showing an example of flavor information output by the information processing apparatus 100.

FIG. 7 is a diagram showing an example of flavor information output by the information processing apparatus 100.

FIG. 8 is a diagram showing an example of a screen output as an evaluation result for each of four types of products (Products A to D).

FIG. 9 is a diagram showing an example of a calculation method for five types of indicators.

FIG. 10 is a diagram showing an example of compounds identified as taste substances.

FIG. 11 is a diagram showing an example of compounds identified as aroma substances.

FIG. 12 is a diagram showing an example of flavor information values for five indicators of four products.

FIG. 13 is a diagram showing an example of reference evaluation values and comparative evaluation values for five indicators of four products.

FIG. 14 is a diagram showing another example of a screen output as an evaluation result for each of four types of products (Products A to D).

FIG. 15 is a flowchart of a main routine performed in the information processing apparatus 100.

FIG. 16 is a flowchart of a subroutine for step S20 in FIG. 15.

FIG. 17 is a flowchart of a subroutine for step S30 in FIG. 15, performed for outputting flavor information (FIGS. 2 to 7).

FIG. 18 is a flowchart of a subroutine for step S30 in FIG. 15, performed for outputting a flavor evaluation (FIGS. 8 and 14).

### DESCRIPTION OF EMBODIMENTS

Hereinafter, embodiments of the present disclosure will be described in detail with reference to the drawings. In the drawings, the same or corresponding parts are denoted by the same reference signs, and a description thereof will not be repeated.

### [1. System Configuration]

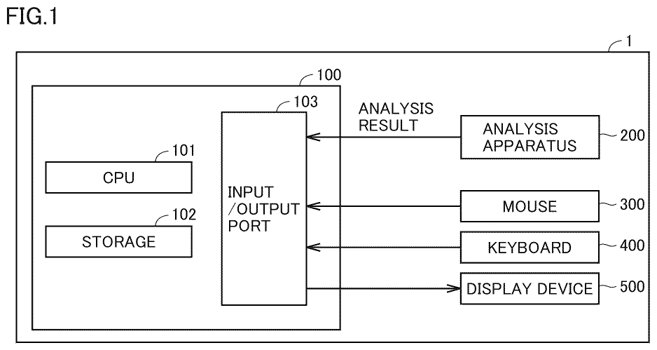

FIG. 1 is a diagram showing a configuration of a flavor information providing system 1. The flavor information providing system 1 mainly includes an information processing apparatus 100 and an analysis apparatus 200. The information processing apparatus 100 acquires an analysis result for a food product from the analysis apparatus 200 and provides information regarding the flavor of the food product using the analysis result. The analysis apparatus 200 may be, for example, a liquid chromatograph-mass spectrometer and/or a gas chromatograph-mass spectrometer.

In one implementation, the information processing apparatus 100 is realized by a general-purpose computer. More specifically, the information processing apparatus 100 includes a CPU (Central Processing Unit) 101, a storage 102, and an input/output port 103.

The CPU 101 is configured by one or more processors. The storage 102 is an example of a storage device and non-transitorily stores a program and/or data. The information processing apparatus 100 acquires an analysis result of a food product from the analysis apparatus 200 via the input/output port 103. The information processing apparatus 100 may be realized by the cooperation of a plurality of computers. In one implementation, the one or more processors constituting the CPU 101 execute a program non-transitorily stored in the storage 102 (or a storage device outside the information processing apparatus 100 accessible by the one or more processors), whereby the information processing apparatus 100 performs various processes.

A mouse 300, a keyboard 400, and a display device 500 are connected to the information processing apparatus 100. The information processing apparatus 100 accepts external input via the mouse 300 and the keyboard 400, and outputs information by displaying a screen on the display device 500. Note that the information processing apparatus 100 may include a network interface and may communicate with an external information device via a network.

In the present embodiment, sake is mainly adopted as an example of a food product. However, the food product for which the information processing apparatus 100 provides information regarding flavor is not limited to sake. Any type of product can be a target for information provision as long as it is a food product to be analyzed by the analysis apparatus 200.

### [2. Flavor Information]

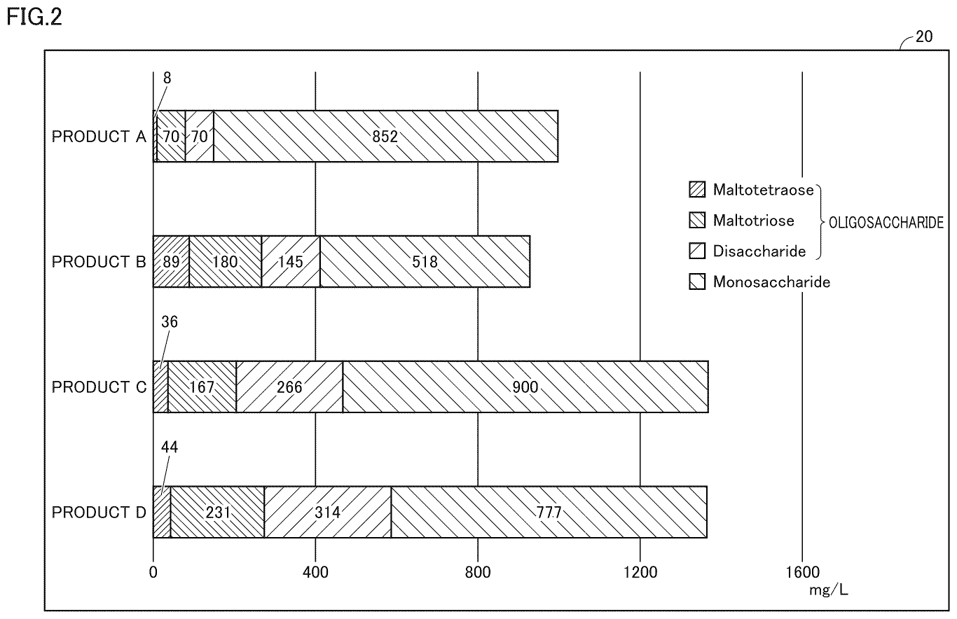

Each of FIGS. 2 to 7 is a diagram showing an example of flavor information output by the information processing apparatus 100.

In one implementation, the information processing apparatus 100 may identify the compounds shown in FIGS. 2 to 7 as substances that affect the difference in flavor among the four types of sake shown in each figure (taste substances or aroma substances), as will be described later. In one implementation, the mass spectrometer outputs the content of each compound in the food product to the information processing apparatus 100 as an analysis result of the food product. More specifically, the mass spectrometer calculates the content of each compound using the ratio of the peak area of each compound to the peak area of an internal standard substance in a mass spectrogram. Note that the content of each compound may be calculated using the concentration of the compound in the sample used for analysis and a calibration curve representing the relationship between concentration and content. Further, the content of each compound may be calculated from the peak area value of a mass spectrogram or a chromatogram.

### (Screen 20 of FIG. 2)

A screen 20 of FIG. 2 displays the content of four types of sugars (Monosaccharide, Disaccharide, Maltotriose, Maltotetraose) for each of four types of sake (Products A, B, C, D).

A user can use the information shown on the screen 20 as support for considering the difference in flavor among Products A, B, C, and D.

More specifically, as shown on the screen 20, Products C and D have a higher total content of the four types of sugars than Products A and B. Note that Products A and B have a higher rice polishing ratio than Products C and D. Therefore, based on the results shown on the screen 20, the user can derive the consideration that products with a lower rice polishing ratio contain more sugars.

As shown on the screen 20, the total content of the four types of sugars in Product A is higher than that in Product B. On the other hand, in Product B, the proportion of three types of oligosaccharides (Disaccharide, Maltotriose, Maltotetraose) in the total sugar is larger than that in Product A. Oligosaccharides have a tendency to be less sweet than monosaccharides. Therefore, the user can derive the consideration that Product B provides a mild sweetness and also reduces irritation from alcohol and acids to provide a mellow mouthfeel.

The screen 20 is an example of flavor information and is an example of taste information output using the values of taste substances.

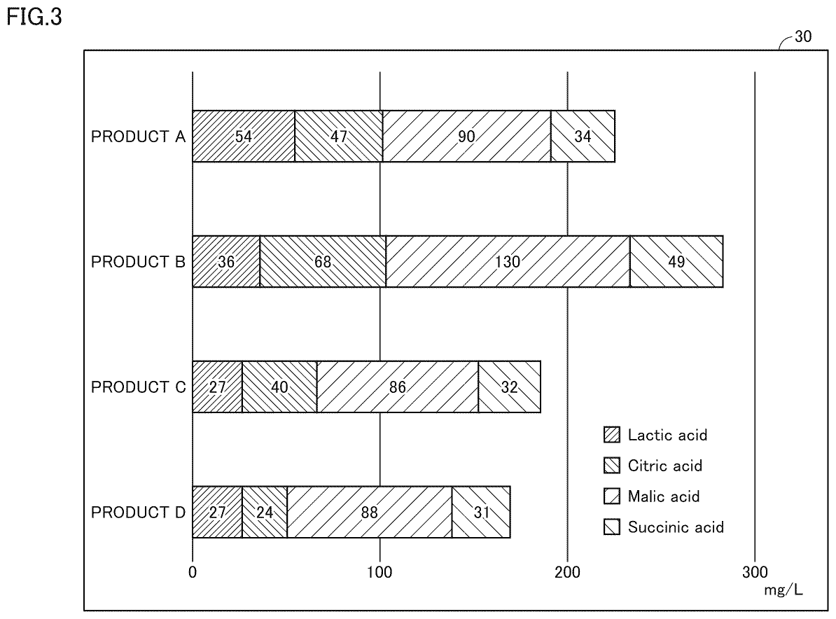

### (Screen 30 of FIG. 3)

A screen 30 of FIG. 3 displays the content of four types of organic acids (Lactic acid, Citric acid, Malic acid, Succinic acid) for each of four types of sake (Products A, B, C, D).

A user can use the information shown on the screen 30 as support for considering the difference in flavor among Products A, B, C, and D.

More specifically, it is said that organic acids in sake are involved in freshness and koku, which can be described as a rich, full-bodied, and deep taste. It is said that the mutual cancellation of sourness and sweetness gives sake a rich taste through their appropriate harmony. In sake, the higher the rice polishing ratio, the relatively more organic acids are contained. Organic acids contribute to the koku of sake. Products C and D (Ginjo-shu, Daiginjo-shu), which showed a tendency to have a high sugar content, have a relatively low organic acid content as shown in FIG. 3. For this reason, it is considered that Products C and D are perceived as sweeter compared to Products A and B.

Also, the quality of sourness differs depending on the type of organic acid. Malic acid and citric acid have a sourness accompanied by a refreshing feeling, and succinic acid also has a unique umami taste. On the screen 30, a tendency is seen in which the content of malic acid is high for all of Products A to D. Therefore, based on the results shown on the screen 30, the user can derive the consideration that all of Products A to D give a clean and refreshing impression.

Further, when the ratio of the content of malic acid to the content of succinic acid is 0.8 or more, it is said that sake has a light aftertaste and a sharp finish (kire). On the screen 30, the ratio of malic acid to succinic acid is 0.8 or more for all of Products A to D. Therefore, based on the results shown on the screen 30, the user can derive the consideration that all of Products A to D have a light aftertaste and a sharp finish.

Furthermore, in Product B, the total value of organic acids including succinic acid is larger than that of the other products. From this, based on the results shown on the screen 30, the consideration can be derived that Product B has the characteristic of having particular umami and koku.

The screen 30 is an example of flavor information and is an example of taste information output using the values of taste substances.

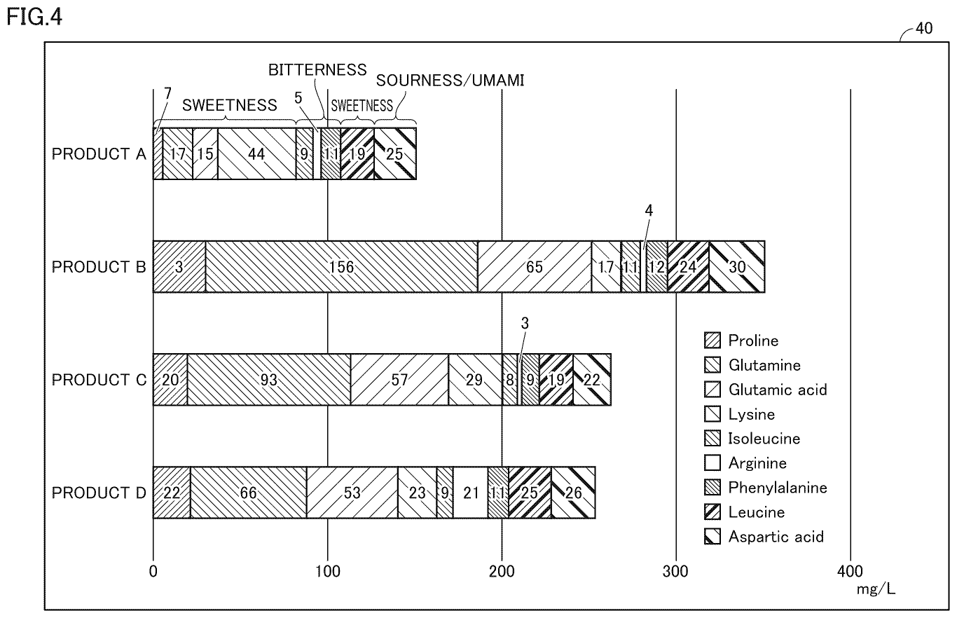

### (Screen 40 of FIG. 4)

A screen 40 of FIG. 4 displays the amino acid content for each of four types of sake (Products A, B, C, D). On the screen 40, the content of the following 10 types of compounds is shown.
Proline
Glutamine
Glutamic acid
Lysine
Isoleucine
Arginine
Phenylalanine
Leucine
Aspartic acid

On the screen 40, a text string representing the type of taste presented by each compound (sweetness, bitterness, sourness/umami) is appended.

A user can use the information shown on the screen 40 as support for considering the difference in flavor among Products A, B, C, and D.

For example, the total amount of amino acid content in Product B significantly exceeds that of the other products. It is said that when the amount of amino acids is large, sake exhibits richness and umami, whereas when it is small, it exhibits a dry (tanrei) taste. Therefore, the user can derive the consideration that Product B exhibits richness and umami, and the remaining products exhibit a dry taste.

Furthermore, Products B to D contain more bitter amino acids (leucine, isoleucine, phenylalanine, and arginine) than Product A. Bitter amino acids give sake sharpness and a dry taste. Therefore, the consideration can be derived that Products B to D provide a flavor that gives more sharpness and a drier taste compared to Product A.

The screen 40 is an example of flavor information and is an example of taste information output using the values of taste substances.

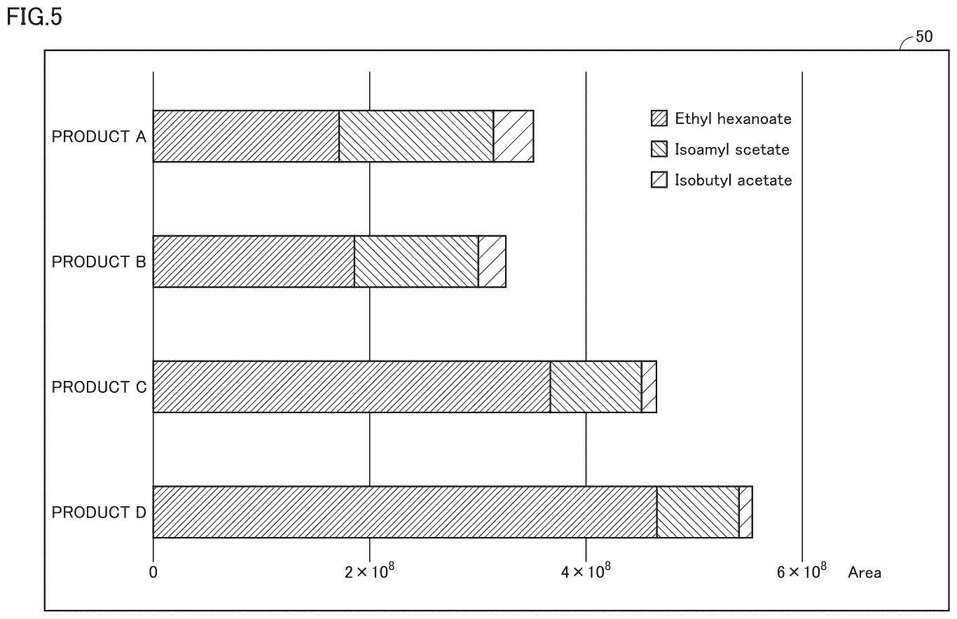

### (Screen 50 of FIG. 5)

A screen 50 of FIG. 5 displays the content of three types of aroma components (Ethyl hexanoate, Isoamyl acetate, Isobutyl acetate) for each of four types of sake (Products A, B, C, D). In one implementation, an aroma component means a volatile substance contained in a food product that has an aroma.

A user can use the information shown on the screen 50 as support for considering the difference in flavor (aroma) among Products A, B, C, and D.

More specifically, the aroma of ethyl hexanoate, which gives a refreshing impression like green apples or pears, and the aromas of isoamyl acetate and isobutyl acetate, like bananas or melons, are called ginjo-ka (ginjo aroma) and are components actually contained in fruits. In Products C and D, ethyl hexanoate accounts for the majority of the aroma components. Therefore, from the screen 50, the user can derive the consideration that Products C and D give a gorgeous impression.

Also, in Products A and B, the ratio of isoamyl acetate and/or isobutyl acetate to ethyl hexanoate is higher than in Products C and D. Therefore, from the screen 50, the user can derive the consideration that Products A and B exhibit an elegant sweet fruit aroma.

The screen 50 is an example of flavor information and is an example of aroma information output using the values of aroma substances.

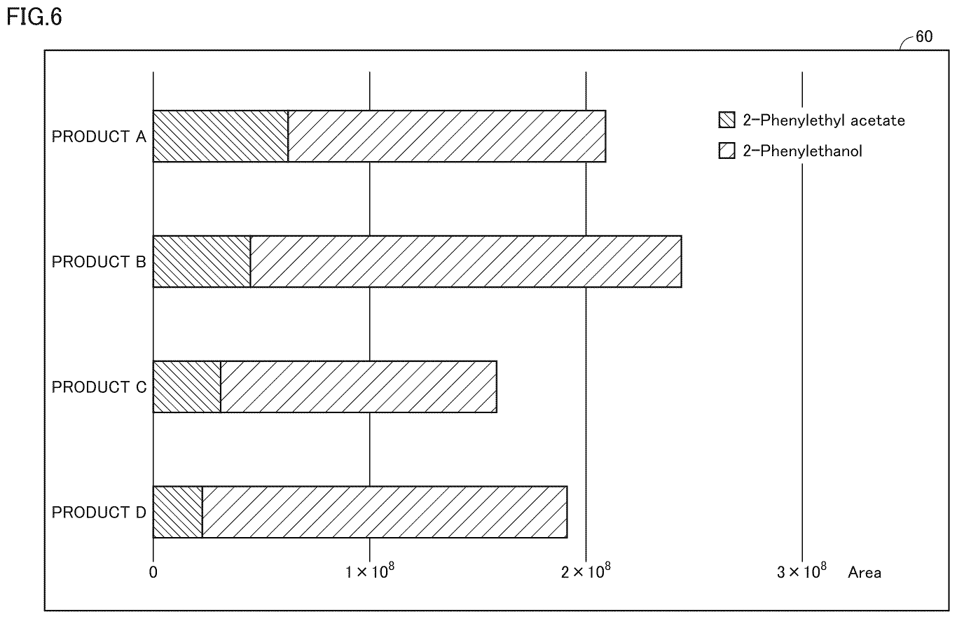

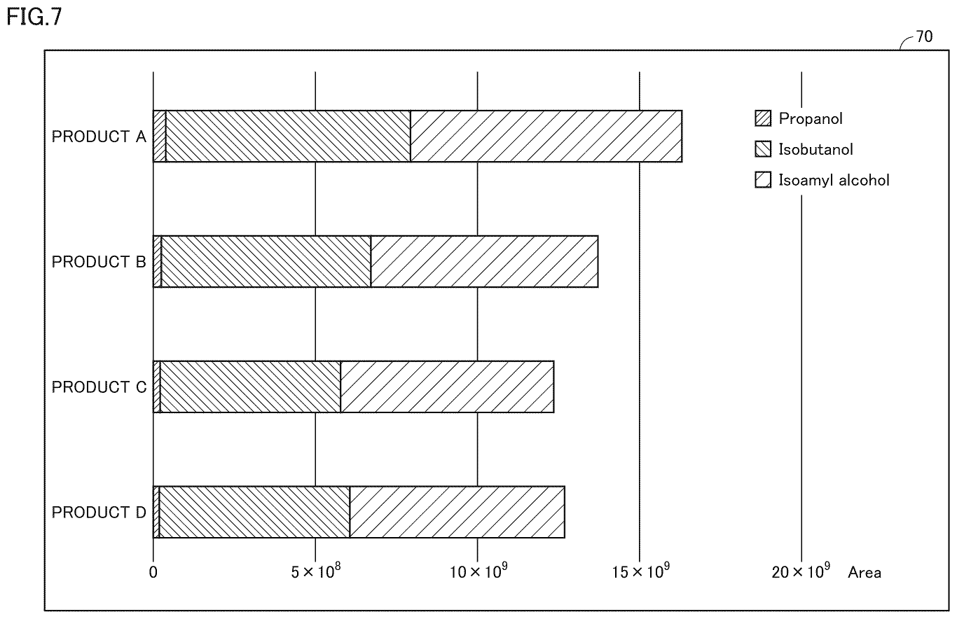

### (Screen 60 of FIG. 6 and Screen 70 of FIG. 7)

A screen 60 of FIG. 6 displays the content of base notes among the aroma components for each of four types of sake (Products A, B, C, D). More specifically, the screen 60 displays the content of two types of base notes: 2-phenylethanol and 2-phenylethyl acetate. A screen 70 of FIG. 7 displays the content of three types of fusel oils (Isoamyl alcohol, Isobutanol, Propanol) for each of four types of sake (Products A, B, C, D).

A user can use the information shown on each of the screen 60 and the screen 70 as support for considering the difference in flavor (aroma) among Products A, B, C, and D.

More specifically, 2-phenylethyl acetate and 2-phenylethanol, which impart a rose-like aroma, are components with a relatively high boiling point that cause a person to perceive an aroma after taking the sake into their mouth. Such a characteristic is referred to as a base note (fukumika). The base note is the source of the sake-like aroma in sake.

As shown on the screen 60, Products A and B have a higher content of base notes than Products C and D. From this, the user can derive from the screen 60 the consideration that Products A and B exhibit a sake-like aroma.

Also, as shown on the screen 70, Products A and B have a higher content of fusel oils than Products C and D. From this, the user can derive from the screen 70 the consideration that Products A and B exhibit a mild aroma.

Each of the screen 60 and the screen 70 is an example of flavor information and is an example of aroma information output using the values of aroma substances.

### [3. Flavor Evaluation]

The information processing apparatus 100 can output, as an evaluation result for each product, an indicator related to the flavor of each product, calculated using one or more analysis results of each product.

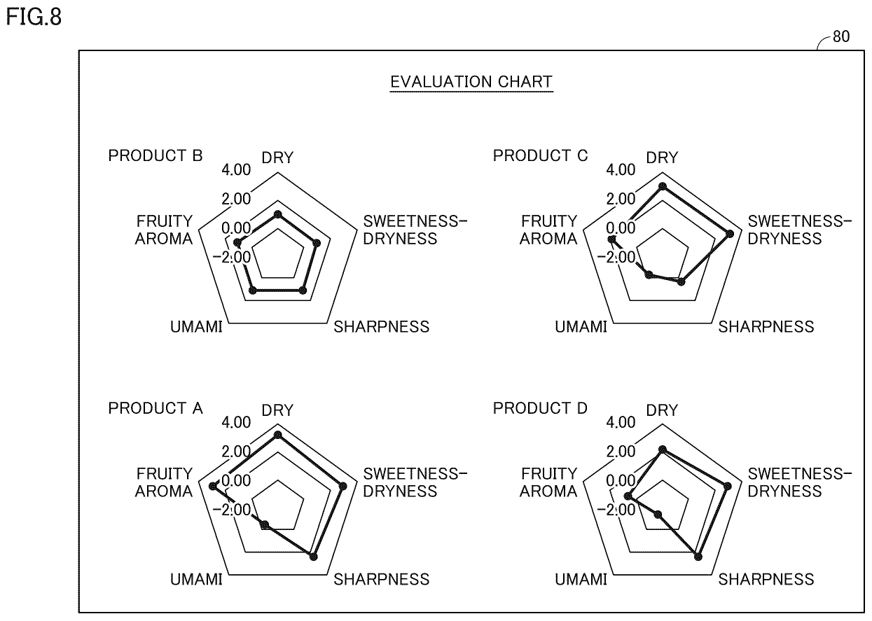

FIG. 8 is a diagram showing an example of a screen output as an evaluation result for each of four types of products (Products A to D). A screen 80 of FIG. 8 includes a radar chart representing the values of five types of indicators (Dry, Sweetness-dryness, Sharpness, Umami, Fruity aroma) for each of Products A to D.

On the screen 80, the radar chart for Product B is shown in the upper left, for Product C in the upper right, for Product A in the lower left, and for Product D in the lower right. On the screen 80, the value of each indicator is normalized such that the value for Product B is 1. That is, the indicator value for each product displayed on the screen 80 is normalized with the indicator value of Product B as a reference. The product used as the normalization reference is also referred to as a “reference product” in this specification. On the screen 80, information identifying the reference product (Product B) may also be displayed.

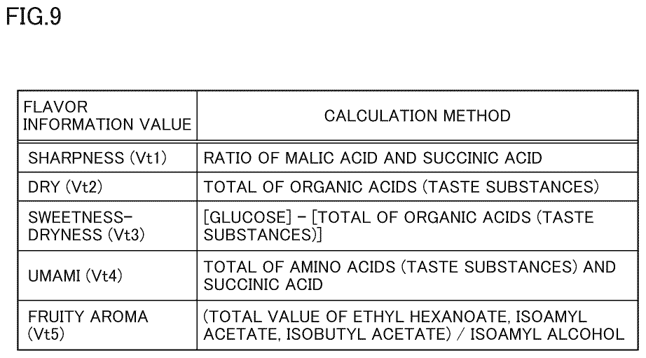

FIG. 9 is a diagram showing an example of a calculation method for five types of indicators. As shown in FIG. 9, the value Vt1 of the indicator “Sharpness” is calculated as the ratio of the content of malic acid to the content of succinic acid in each product.

The value Vt2 of the indicator “dry” is calculated as the total content of substances identified as “taste substances” among organic acids in each product, which will be described later. The lower the content of organic acids, the dryer (tanrei) the sake is said to be. From this, it is considered that the smaller the value Vt2 of a certain sake, the higher the degree to which that sake is dry.

Conventionally, the content of all organic acids was sometimes used as an indicator of flavor. On the other hand, in the present embodiment, as taste substances, some organic acids are identified as taste substances that affect the difference in flavor among Products A to D among the organic acids. That is, only the content of some organic acids is used as the content of organic acids that affect the difference in flavor among Products A to D. As a result, the value Vt2 of the indicator “Dry” is calculated as a value that more accurately represents the difference among Products A to D.

The value Vt3 of the indicator “Sweetness-dryness” is calculated by subtracting the content of substances identified as “taste substances” among organic acids, which will be described later, from the content of glucose in each product. As described above, in sake, sourness and sweetness cancel each other out, thereby moderately harmonizing the taste. From this, it is considered that the larger the value Vt3 of a certain sake, the more sweetness it has, and the smaller it is, the more dryness it has.

For the calculation of the value Vt3, similar to the calculation of the value Vt2, the content of substances identified as taste substances among organic acids is used. As a result, the value Vt3, similar to the value Vt2, is calculated as a value that more accurately represents the difference among Products A to D.

The value Vt4 of the indicator “Umami” is calculated as the sum of the content of substances identified as “taste substances” among amino acids, which will be described later, and the content of succinic acid in each product.

Conventionally, the content of all amino acids was sometimes used as an indicator of flavor. On the other hand, in the present embodiment, as taste substances, some amino acids are identified as taste substances that affect the difference in flavor among Products A to D among the amino acids. That is, only the content of some amino acids is used as the content of amino acids that affect the difference in flavor among Products A to D. As a result, the value Vt4 of the indicator “Umami” is calculated as a value that more accurately represents the difference among Products A to D.

The value Vt5 of the indicator “Fruity aroma” is calculated as the value obtained by dividing the total content of three types of aroma components (ethyl hexanoate, isoamyl acetate, and isobutyl acetate) by the content of isoamyl alcohol in each product. Ethyl hexanoate, isoamyl acetate, and isobutyl acetate are identified as aroma substances among the aroma components, as will be described later.

Conventionally, a value obtained by dividing the content of ethyl hexanoate by the content of isoamyl alcohol was sometimes used as an indicator of fruity aroma. On the other hand, in the present embodiment, in addition to ethyl hexanoate, isoamyl acetate and isobutyl acetate can be identified as aroma substances that affect the difference in flavor among Products A to D. That is, as the content of aroma components that affect the difference in flavor among Products A to D, the content of more types of substances than conventionally is used. As a result, the value Vt5 of the indicator “Fruity aroma” is calculated as a value that more accurately represents the difference among Products A to D.

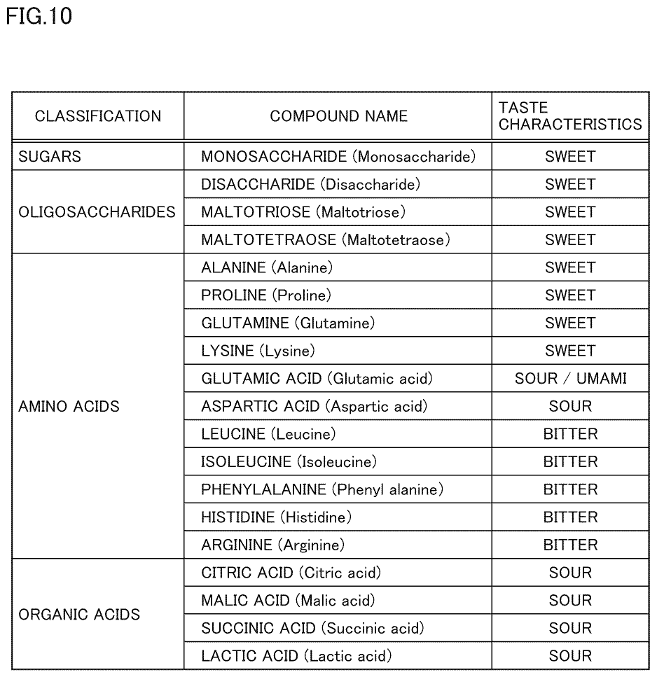

FIG. 10 is a diagram showing an example of compounds identified as taste substances. In FIG. 10, compounds are shown along with their classification and taste characteristics. In one implementation, when the information processing apparatus 100 identifies a taste substance, it may acquire the taste characteristics of each taste substance by searching a database that associates substances with taste characteristics, generate a table as shown in FIG. 10, and store the table in the storage 102.

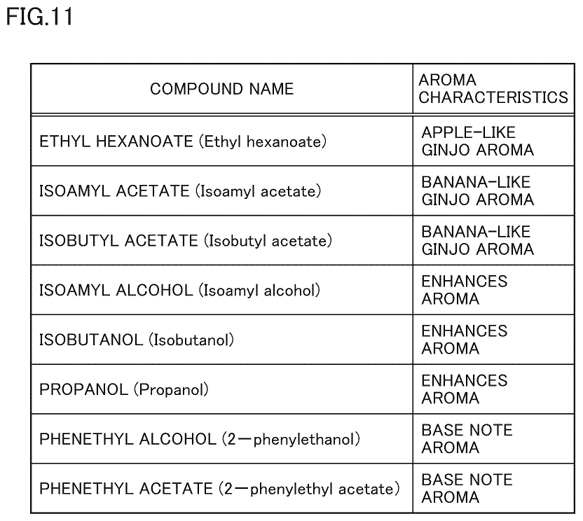

FIG. 11 is a diagram showing an example of compounds identified as aroma substances. In FIG. 11, compounds are shown along with their classification and aroma characteristics. In one implementation, when the information processing apparatus 100 identifies an aroma substance, it may acquire the aroma characteristics of each aroma substance by searching a database that associates substances with aroma characteristics, generate a table as shown in FIG. 11, and store the table in the storage 102.

The calculation method for each of the five types of indicators is not limited to that shown in FIG. 9. The user may appropriately change the calculation method for the five types of indicators by referring to the types and/or taste or aroma characteristics of the substances identified as taste substances and aroma substances. Further, the number of indicators to be calculated is not limited to five. It may be one type, or any number of two or more types. [4. Identification of Taste Substances] The identification of taste substances will be described.

The information processing apparatus 100 acquires an analysis result of taste components for each of Products A to D. A taste component, also called a flavor component, means a component that provides some kind of taste.

The analysis result represents the content of each of two or more components. For example, when a certain amount of each product is introduced into a liquid chromatograph-mass spectrometer (analysis apparatus 200), the content of each component is derived as the ratio of the peak area of each component to the peak area of an internal standard substance. The information processing apparatus 100 acquires the content of each component from the analysis apparatus 200 as the analysis result of the taste components.

For example, when the content of 151 types of hydrophilic metabolites (sugars, amino acids, organic acids, nucleosides, nucleotides, etc.) is acquired as the analysis result of the taste components of each product, the analysis result of the four types of products includes 604 measured values (contents).

The information processing apparatus 100 performs an ANOVA (analysis of variance) test on the analysis results of the four types of products. Then, the information processing apparatus 100 identifies, as taste substances, the components corresponding to the measured values having a p-value of 0.05 or more in the result of this test. An example of the taste substances to be identified is the compounds listed in FIG. 10. As a result, among the plurality of components constituting the flavor, the components that particularly affect the difference in flavor can be identified.

Note that the information processing apparatus 100 employs the ANOVA test as a test for the analysis results of three or more types of products, and employs the Student's t-test or the Mann-Whitney U-test as a test for the analysis results of two types of products.

The information processing apparatus 100 may acquire the analysis results used for identifying the taste substances from a gas chromatograph-mass spectrometer.

The components of the taste substances to be identified may be limited to only substances included in a component group predetermined for each flavor. For example, for the flavor “Dry” components other than organic acids may not be used as taste substances. As a result, components that have a small contribution to the flavor can be removed from the basis of the flavor information.

### [5. Identification of Aroma Substances]

The identification of aroma substances will be described.

The information processing apparatus 100 acquires an analysis result of aroma components for each of Products A to D. The analysis result represents the content of each of two or more components. For example, when a certain amount of each product is introduced into a gas chromatograph-mass spectrometer (analysis apparatus 200), the content of each component is derived as the ratio of the peak area of each component to the peak area of an internal standard substance. The information processing apparatus 100 acquires the content of each component from the analysis apparatus 200 as the analysis result of the aroma components.

The information processing apparatus 100 performs an ANOVA (analysis of variance) test on the analysis results of the four types of products. Then, the information processing apparatus 100 identifies, as aroma substances, the components corresponding to the measured values having a p-value of 0.05 or more in the result of this test. An example of the aroma substances to be identified is the compounds listed in FIG. 11. As a result, among the plurality of components constituting the flavor, the components that particularly affect the difference in flavor can be identified.

Note that the information processing apparatus 100 employs the ANOVA test as a test for the analysis results of three or more types of products, and employs the Student's t-test or the Mann-Whitney U-test as a test for the analysis results of two types of products.

The information processing apparatus 100 may acquire the analysis results used for identifying the aroma substances from a liquid chromatograph-mass spectrometer.

The components of the aroma substances to be identified may be limited to only substances included in a component group predetermined for each flavor. For example, for the flavor “Dry” components other than organic acids may not be used as aroma substances. As a result, components that have a small contribution to the flavor can be removed from the basis of the flavor information.

### [6. Flavor Information Value]

The information processing apparatus 100 calculates the value of an indicator as shown in FIG. 9 as the flavor information value of each product. That is, the respective values of the indicators Vt1 to Vt5 constitute an example of a flavor information value.

### [7. Reference Evaluation Value and Comparative Evaluation Value]

“Reference evaluation value” and “comparative evaluation value” are defined as values used for the radar chart as shown in FIG. 8. Hereinafter, these values will be described.

When generating the radar chart as shown in FIG. 8, the information processing apparatus 100 adjusts the flavor information values so that the indicator values of one product among the plurality of types of products all become 1. For this purpose, the information processing apparatus 100 performs a common operation on the flavor information values of all products.

More specifically, it is assumed that when the value of the indicator Vt1 of Product B is P1b, an operation f(x) for making the value of P1b equal to 1 is specified. In this case, f(P1b)=1. Such an operation is performed on the respective values P1a, P1c, and P1d of the indicator Vt1 of Products A, C, and D. It is assumed that by this operation, the respective values P1a, P1c, and P1d of the indicator Vt1 of Products A, C, and D are converted into values N1a, N1c, and N1d. At this time, on the radar chart, 1, N1a, N1c, and N1d are respectively posted as the respective values of the indicator Vt1 of Products B, A, C, and D.

For a more specific explanation, assume 3, 4, 2, and 3 as the respective values (flavor information values) of the indicator Vt1 of Products B, A, C, and D. As an example of an operation for making the flavor information value of Product B equal to 1, an operation of “subtracting 2” is specified. When this operation is performed on the respective flavor information values of Products A, C, and D, 2, 0, and 1 are derived as the respective values of N1a, N1c, and N1d. In this case, on the radar chart, 1, 2, 0, and 1 are respectively posted as the respective values of the indicator Vt1 of Products B, A, C, and D.

The “reference evaluation value” means the value posted on the radar chart for the product used as a reference. In the above example, it is the value for Product B, which is 1.

The “comparative evaluation value” means the value formed on the radar chart for products other than the product used as a reference. In the above example, they are the respective values for Products A, C, and D, which are 2, 0, and 1, respectively. As a result, the “comparative evaluation value” means a value normalized with respect to the flavor evaluation value of Product B.

Note that the operation for normalizing the flavor information value is also referred to as “adjustment information” in this specification. The adjustment information is not limited to addition or subtraction, and any function may be adopted as the adjustment information.

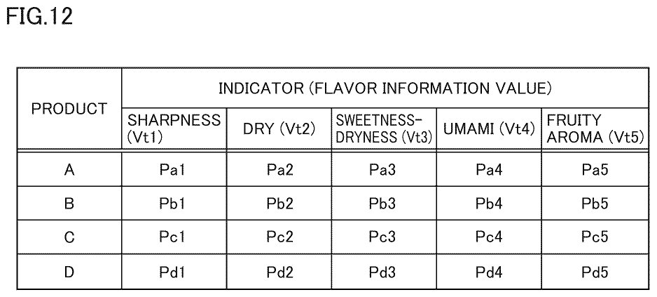

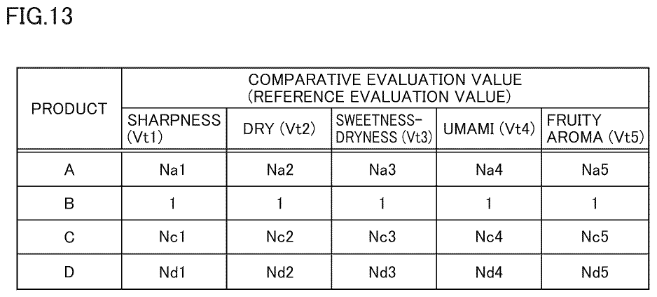

FIG. 12 is a diagram showing an example of flavor information values for five indicators of four products. FIG. 13 is a diagram showing an example of reference evaluation values and comparative evaluation values for five indicators of four products. The derivation of the reference evaluation values and the comparative evaluation values will be described more specifically with reference to FIGS. 12 and 13.

As shown in FIG. 12, the information processing apparatus 100 calculates the flavor information values of five types of indicators (Vt1 to Vt5) for each of the four products.

Then, as shown in FIG. 13, the information processing apparatus 100 converts the five types of flavor information values of one product (Product B) among the four products into reference information values using a given function. For each of the five types of indicators (Vt1 to Vt5), a separate function is used for the conversion from the flavor information value to the reference information value.

Then, the information processing apparatus 100 converts the five types of flavor information values into comparative evaluation values for each of the remaining three products. For example, for the conversion from the flavor information value of the indicator Vt1 to the comparative evaluation value, the function for the indicator Vt1 is used. For the conversion from the respective flavor information values of the indicators Vt2 to Vt5 to the comparative evaluation values, the respective functions for the indicators Vt2 to Vt5 are used.

### [8. Identification of Similar Products]

The information processing apparatus 100 may identify a product having a similar flavor for a certain product.

In one example, the information processing apparatus 100 identifies, as a product having a similar flavor, the product for which the difference (the sum of the differences) between the reference evaluation value and the comparative evaluation value for one or more indicators is the minimum.

In the example shown in FIG. 13, the sums Sa, Sc, and Sd of the differences between the reference evaluation value and the comparative evaluation value for each of Products A, C, and D are calculated according to the following equations (1) to (3).

$$Sa = ( 1 - Na 1 ) + ( 1 - Na 2 ) + ( 1 - Na 3 ) + ( 1 - Na 4 ) + ( 1 - Na 5 ) \qquad ( 1 )$$

$$Sc = ( 1 - Nc 1 ) + ( 1 - Nc 2 ) + ( 1 - Nc 3 ) + ( 1 - Nc 4 ) + ( 1 - Nc 5 ) \qquad ( 2 )$$

$$Sd = ( 1 - Nd 1 ) + ( 1 - Nd 2 ) + ( 1 - Nd 3 ) + ( 1 - Nd 4 ) + ( 1 - Nd 5 ) \qquad ( 3 )$$

The information processing apparatus 100 may identify, as a similar product to Product B, the product among Products A, C, and D for which the value of the above difference is the smallest.

The information processing apparatus 100 may also identify, as a similar product to Product B, the product among Products A, C, and D for which the absolute value of the above difference is the smallest.

The information processing apparatus 100 may also identify, as a similar product to Product B, a product among Products A, C, and D for which the value (or absolute value) of the above difference is smaller than a given threshold.

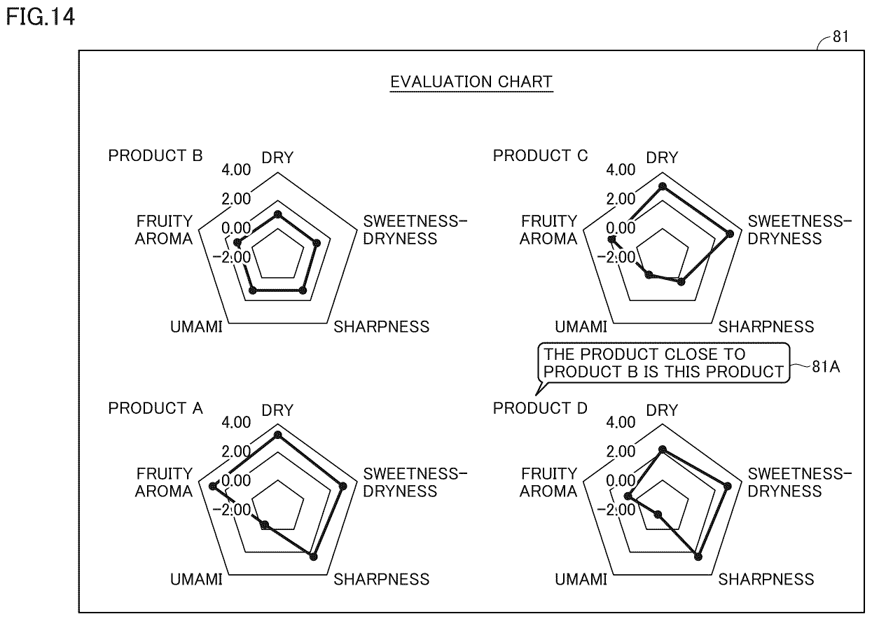

FIG. 14 is a diagram showing another example of a screen output as an evaluation result for each of four types of products (Products A to D). The information processing apparatus 100 may display information indicating the product identified as a similar product. A screen 81 of FIG. 14 further includes a message 81A with respect to the screen 80 of FIG. 8. In the example of FIG. 14, the message 81A includes the text string “The product close to Product B is this product.”

Note that if all the values (or absolute values) of the sums Sa, Sc, and Sd exceed a specific threshold, the information processing apparatus 100 may determine that there is no similar product to Product B among Products A, C, and D. When the information processing apparatus 100 determines that there is no similar product, it may output a screen in which information indicating that there is no similar product to Product B is added to the screen 80.

### [9. Processing Flow]

### (Main Routine)

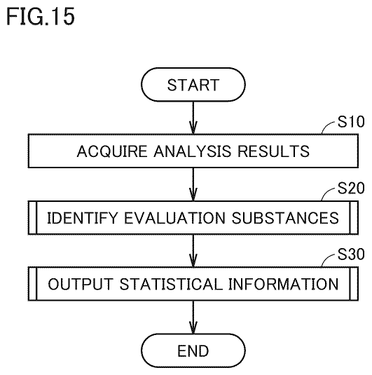

FIG. 15 is a flowchart of a main routine performed in the information processing apparatus 100. In one implementation, the information processing apparatus 100 starts the process of FIG. 15 in response to receiving a start instruction from a user. In one implementation, in the information processing apparatus 100, the one or more processors constituting the CPU 101 execute a given program, thereby realizing the processes described in this specification.

In step S10, the information processing apparatus 100 acquires an analysis result for each of two or more food products from the analysis apparatus 200.

In step S20, the information processing apparatus 100 identifies an evaluation substance. The evaluation substance is a general term for the aforementioned taste substances and aroma substances.

In step S30, the information processing apparatus 100 outputs statistical information of the evaluation substance among the analysis results acquired in step S10, and ends the process of FIG. 15.

### (Identify Evaluation Substance)

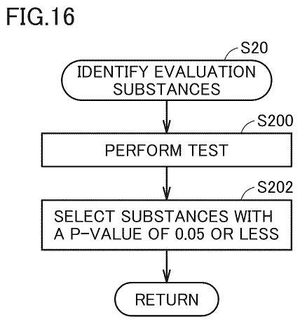

FIG. 16 is a flowchart of a subroutine for step S20 in FIG. 15.

In step S20, in step S200, the information processing apparatus 100 performs a test (the aforementioned ANOVA or Student's t-test or Mann-Whitney U-test) on the analysis results acquired in step S10. In step S200, the information processing apparatus 100 may perform the test for each of the taste components and the aroma components. That is, the information processing apparatus 100 may perform the test on the analysis result of the taste components, and may also perform the test on the analysis result of the aroma components.

In step S202, the information processing apparatus 100 identifies a substance based on the p-value which is the result of the test in step S200. More specifically, the information processing apparatus 100 identifies a substance having a p-value of 0.05 or less in the result of the test as a taste substance or an aroma substance. Thereafter, the information processing apparatus 100 returns the control to FIG. 15.

### (Output Statistical Information-Output of Flavor Information)

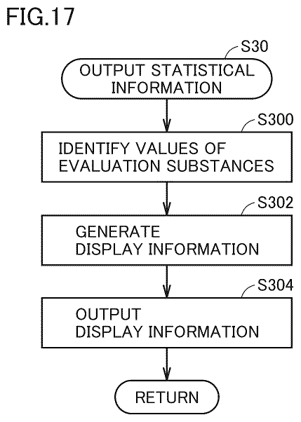

FIG. 17 is a flowchart of a subroutine for step S30 in FIG. 15, performed for outputting flavor information (FIGS. 2 to 7). In one implementation, an instruction for performing the output of flavor information is input to the information processing apparatus 100. In response to the input of the instruction, the information processing apparatus 100 performs the subroutine of FIG. 17.

In step S30, in step S300, the information processing apparatus 100 identifies the value of the evaluation substance. More specifically, the information processing apparatus 100 extracts the respective analysis results of one or more evaluation substances identified in step S20 from the analysis results acquired in step S10.

In step S302, the information processing apparatus 100 generates screen information (display information) to be displayed as flavor information.

In step S304, the information processing apparatus 100 outputs the display information generated in step S302 to the display device 500. As a result, the screen described with reference to FIG. 2, etc., is displayed as flavor information. Thereafter, the information processing apparatus 100 returns the control to FIG. 15.

Note that the output to the display device 500 is one mode of outputting flavor information. The flavor information may be output in a mode other than display (for example, audio).

### (Output Statistical Information-Output of Flavor Evaluation)

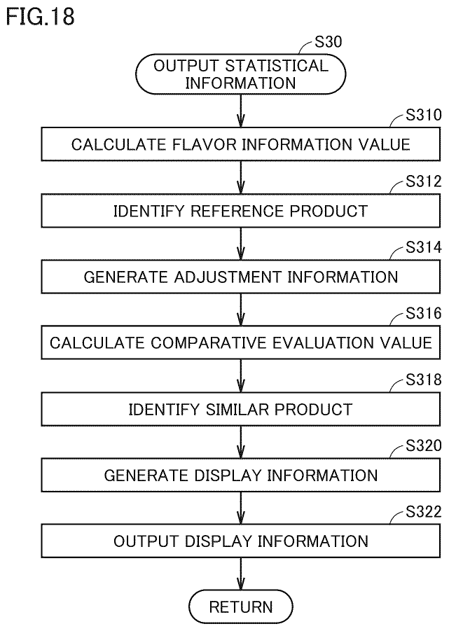

FIG. 18 is a flowchart of a subroutine for step S30 in FIG. 15, performed for outputting a flavor evaluation (FIGS. 8 and 14). In one implementation, an instruction for performing the output of a flavor evaluation is input to the information processing apparatus 100. In response to the input of the instruction, the information processing apparatus 100 performs the subroutine of FIG. 18. The subroutine of FIG. 18 may be performed in parallel with the subroutine of FIG. 17, or before or after the subroutine of FIG. 17.

In step S30, in step S310, the information processing apparatus 100 calculates a flavor information value of one or more indicators for each of the two or more food products.

In step S312, the information processing apparatus 100 identifies a reference product. In one implementation, the reference product is specified by the user. The information processing apparatus 100 identifies a reference product from the two or more food products in response to the input of a specification from the user.

In step S314, the information processing apparatus 100 generates adjustment information (operation for normalization). The adjustment information is information representing an operation for making the flavor information value of the reference product equal to 1.

In step S316, the information processing apparatus 100 calculates a comparative evaluation value (FIG. 13) for the food products other than the reference product among the two or more food products.

In step S318, the information processing apparatus 100 identifies a similar product to the reference product.

In step S320, the information processing apparatus 100 generates display information for outputting a flavor evaluation (for example, screen information for displaying the screen 80 of FIG. 8 or the screen 81 of FIG. 14).

In step S322, the information processing apparatus 100 outputs the display information generated in step S320 to the display device 500. As a result, the screen described with reference to FIG. 8 or FIG. 14 is displayed as a flavor evaluation. Thereafter, the information processing apparatus 100 returns the control to FIG. 15.

Note that the output to the display device 500 is one mode of outputting a flavor evaluation. The flavor evaluation may be output in a mode other than display (for example, audio).

According to the embodiment described above, for each of two or more food products, the respective information values of one or more flavors are output in a state of being normalized with one food product as a reference. As a result, information regarding the flavor of two or more food products is provided as relative information, and thereby, an accurate evaluation of the two or more food products is provided.

### [Aspects]

It will be understood by those skilled in the art that the plurality of exemplary embodiments described above are specific examples of the following aspects.

(Item 1) A method according to one aspect is a method for providing an evaluation of a flavor of two or more food products, the method comprising: a step of acquiring, for each of the two or more food products, respective information values for one or more flavors; a step of generating, for one food product selected from the two or more food products, adjustment information for making the information values a reference for normalization; a step of normalizing the respective information values of the food products other than the one food product among the two or more food products by using the adjustment information; and a step of outputting, for each of the two or more food products, the normalized values of the information values.

According to the method described in Item 1, it becomes easy to perform a comparative evaluation of the flavor of a plurality of food products.

(Item 2) In the method described in Item 1, the step of outputting the normalized values of the information values may include displaying the normalized values of the information values as one graph for each of the two or more food products.

According to the method described in Item 2, the provided evaluation is provided in an easy-to-view manner.

(Item 3) In the method described in Item 2, the graph may be a radar chart.

According to the method described in Item 3, when the provided evaluation includes multiple types of information values, it is provided in an easy-to-view manner.

(Item 4) The method according to any one of Items 1 to 3 may further comprise a step of acquiring the content of taste substances and aroma substances for each of the two or more food products, wherein the step of acquiring the respective information values for the one or more flavors may include calculating the information values using the content of the taste substances and the content of the aroma substances.

According to the method described in Item 4, an accurate evaluation of the flavor of a food product is provided for each of the taste components and aroma components regarding the flavor of the food product.

(Item 5) In the method according to any one of Items 1 to 4, the content of the taste substances and the aroma substances may be measured using a liquid chromatograph or a gas chromatograph.

According to the method described in Item 5, accurate values are obtained as analysis results of the taste components and aroma components.

(Item 6) In the method according to any one of Items 1 to 5, the content of the taste substances may be measured using a liquid chromatograph, and the content of the aroma substances may be measured using a gas chromatograph.

Since many taste substances are substances that are liquid or solid at room temperature, and many aroma substances are substances that are gaseous at room temperature, according to the method described in Item 6, accurate values are obtained as analysis results of the taste components and aroma components.

(Item 7) The method according to Item 4 may further comprise a step of identifying, from the components, the taste substances and the aroma substances as substances that affect the difference in flavor among the two or more food products, using a result of a test that uses the analysis result of the components of each of the two or more food products.

According to the method described in Item 7, the evaluation of the flavor of two or more food products is based on substances that affect the difference in flavor among the two or more food products. As a result, the evaluation of the flavor of the two or more food products becomes more accurate.

(Item 8) The method according to any one of Items 1 to 7 may further comprise a step of identifying, from among the two or more food products, a food product having a flavor similar to the one food product, based on the normalized values of the respective information values of the two or more food products.

According to the method described in Item 8, easy-to-understand information is provided as to which food product is similar to the one food product used as the normalization reference among the two or more food products.

(Item 9) An information processing apparatus according to one aspect may comprise: one or more processors; and a storage device storing a program that, when executed by the one or more processors, causes the one or more processors to perform the method according to any one of Items 1 to 8.

According to the information processing apparatus described in Item 9, an accurate evaluation of the flavor of a food product is provided to general consumers.

(Item 10) A flavor information providing system according to one aspect may comprise: the information processing apparatus according to Item 9; and an analysis apparatus that outputs an analysis result of a food product to the information processing apparatus.

According to the flavor information providing system described in Item 10, an accurate evaluation of the flavor of a food product is provided to general consumers.

(Item 11) A program according to one aspect may, when executed by one or more processors, cause the one or more processors to perform the method according to any one of Items 1 to 8.

According to the program described in Item 11, an accurate evaluation of the flavor of a food product is provided to general consumers.

The embodiments disclosed herein should be considered in all respects as illustrative and not restrictive. The scope of the present disclosure is indicated by the claims rather than by the description of the embodiments above, and all changes that come within the meaning and range of equivalency of the claims are intended to be embraced therein. Further, it is intended that the respective technologies in the embodiments can be implemented alone or in combination with other technologies in the embodiments as much as possible, as necessary.

### REFERENCE SIGNS LIST

1 Flavor information providing system, 20, 30, 40, 50, 60, 70, 80, 81 Screen, 81A Message, 100 Information processing apparatus, 101 CPU, 102 Storage, 103 Input/output port, 200 Analysis apparatus, 300 Mouse, 400 Keyboard, 500 Display device.

1. A method for providing an evaluation of a flavor of two or more food products, the method comprising:
a step of acquiring, for each of the two or more food products, respective information values for one or more flavors;
a step of generating, for one food product selected from the two or more food products, adjustment information for making the information values a reference for normalization;
a step of normalizing the respective information values of the food products other than the one food product among the two or more food products by using the adjustment information; and
a step of outputting, for each of the two or more food products, the normalized values of the information values.

2. The method for providing an evaluation of a flavor according to claim 1, wherein the step of outputting the normalized values of the information values includes displaying the normalized values of the information values as one graph for each of the two or more food products.

3. The method for providing an evaluation of a flavor according to claim 2, wherein the graph is a radar chart.

4. The method for providing an evaluation of a flavor according to claim 1, further comprising:
a step of acquiring the content of taste substances and aroma substances for each of the two or more food products,
wherein the step of acquiring the respective information values for the one or more flavors includes calculating the information values using the content of the taste substances and the content of the aroma substances.

5. The method for providing an evaluation of a flavor according to claim 4, wherein the content of the taste substances and the aroma substances is measured using a liquid chromatograph or a gas chromatograph.

6. The method for providing an evaluation of a flavor according to claim 4, wherein the content of the taste substances is measured using a liquid chromatograph, and
the content of the aroma substances is measured using a gas chromatograph.

7. The method for providing an evaluation of a flavor according to claim 4, further comprising:
a step of identifying, from the components, the taste substances and the aroma substances as substances that affect the difference in flavor among the two or more food products, using a result of a test that uses an analysis result of the components of each of the two or more food products.

8. The method for providing an evaluation of a flavor according to claim 1, further comprising:
a step of identifying, from among the two or more food products, a food product having a flavor similar to the one food product, based on the normalized values of the respective information values of the two or more food products.

9. An information processing apparatus, comprising:
one or more processors; and
a storage device storing a program that, when executed by the one or more processors, causes the one or more processors to perform the method according to claim 1.

10. A flavor information providing system, comprising:
the information processing apparatus according to claim 9; and
an analysis apparatus that outputs an analysis result of a food product to the information processing apparatus.

11. A non-transitory computer readable medium storing a program that, when executed by one or more processors, causes the one or more processors to perform the method according to claim 1.
